(19) United States
(12) Patent Application Publication
(10) Pub. No.: US 2019/0184076 A1
(43) Pub. Date: Jun. 20, 2019

(54) IMPROVEMENTS IN OR RELATING TO BLOOD SALVAGE AND AUTOTRANSFUSION

(71) Applicant: UNIVERSITY OF STRATHCLYDE, Glasgow (GB)

(72) Inventor: Terence GOURLAY, Renfrewshire (GB)

(21) Appl. No.: 16/331,399

(22) PCT Filed: Sep. 7, 2017
(86) PCT No.: PCT/GB2017/052610

§ 371 Date: Mar. 7, 2019

(30) Foreign Application Priority Data
Sep. 7, 2016 (GB) 1615189.6

(51) Int. Cl. A61M 1/02; F04B 43/12; B01D 61/20; A61M 1/38; B01D 61/18; F04B 9/14; A61M 1/10; A61M 1/36

(52) U.S. Cl. CPC A61M 1/0281; A61M 1/0218; F04B 43/1292; F04B 43/1276; B01D 61/20; B01D 2313/243; B01D 61/18; F04B 9/14; A61M 1/1046; A61M 1/1039; A61M 1/3627; A61M 1/38

(57) ABSTRACT

Apparatus for blood processing comprising a blood collection reservoir (30), a blood collection conduit, a blood transfer conduit, and a manually operable pump unit (10) comprising first and second peristaltic pumps (10A, 10B). The first peristaltic pump is mounted about and acts upon the blood collection conduit. The second peristaltic pump is mounted about and acts upon the blood transfer conduit. The pump unit is provided with an actuator (112) adapted to switch the second peristaltic pump between an operative condition and an inoperative condition: in the operative condition the second peristaltic pump is engaged with the blood transfer conduit to convey blood from the blood collection reservoir while the first peristaltic pump is engaged with the blood collection conduit to convey blood from a wound site to the blood collection reservoir, and in the inoperative condition the second peristaltic pump is disengaged from the blood transfer conduit.

### FIELD OF THE INVENTION

This invention relates to improved apparatus for salvage and autotransfusion of blood evacuated from wound sites. The apparatus is of particular utility in austere situations in which, for instance, electrical power is not available. As such, the apparatus may be useful in military applications, including battlefield medical facilities.

### BACKGROUND TO THE INVENTION

Uncontrolled haemorrhage is the leading cause of death on the battlefield, where two thirds of these deaths are a result of non-compressible (truncal) haemorrhage. In the recent conflicts in Iraq and Afghanistan, uncontrolled blood loss was the cause of death in 85% of the potentially survivable battlefield cases and in 80% of those who died in a military treatment facility. Between 2001 and 2011 in these war zones, 25% of the 4596 combat deaths were potentially survivable, and 90% of the deaths occurred before the injured reached a medical facility. Of the 4090 mortally wounded troops 1391 died instantly and 2699 died before reaching a treatment facility—only 506 reached a hospital before succumbing to injury. This data highlights an obvious need for improving field treatment capability.

Over 25% of military casualties require aggressive and urgent haemostatic resuscitation to increase survivability. Additionally, 30-40% of these military personnel that need blood transfusions when they reach emergency departments also suffer from acute traumatic coagulopathy (ATC), which is associated with an 80% mortality rate. Variations of blood transfusion techniques are possible, although very few blood restoration modalities are available to a field medic in austere conditions, due to blood provision and storage constraints. The urgent deployment of blood products (fresh frozen plasma and packed red blood cells) has been shown to attenuate ATC, although there are considerable logistical concerns and clinical risk with this type of intervention. For these reasons, there is a need to develop innovative and less logistically intense mechanisms for treating casualties that require blood at point of injury.

More recent damage control resuscitation (DCR) protocols have modified the ratios of blood elements administered to war casualties and signalled the necessary implementation of whole fresh warm blood (WFWB) administration in combat hospitals, where ‘buddy transfusions’ have exceeded 6000 units in recent combat operations. Although WFWB transfusions in the battlefield are feasible—having been shown to aid effectiveness of clotting and oxygen transport—there is still potential for catastrophic outcomes such as haemolytic reactions. Also, donors are unable to provide more than a single unit, require fluid therapy and are limited to light duty for at least 72 hours following donation, and are restricted from flying duties for this time period.

Military blood transfusion relies on donations from the general public and military personnel for treating massive blood loss in casualties. In 2013, 8000 blood components from UK donors were shipped overseas to combat zones, which required safe and sterile transportation over thousands of miles to military hospitals. Donated blood has been shown to increase death rates, accentuate bleeding, and carries risk of biological reactions. Worryingly, transfusion reactions are difficult to recognise in severely or multiply injured casualties. Haemolytic reactions present acutely with fever, dyspnea and renal failure and delayed reactions may occur. These deleterious consequences are avoided where the patient's own blood is collected and recycled—a process known as auto-transfusion.

An auto-transfusion device known as HemoSep® is already known for blood cell salvage in non-military clinical applications. HemoSep® is a portable intraoperative blood salvage device that achieves effective blood recycling—concentrating the residual blood during and after an operative procedure, returning all cell species including platelets and red blood cells back to the patient—and it has been successfully and broadly used in a wide range of applications. The concentrate comprises all cell types, including platelets, which are crucial to haemostasis but which are not salvaged by alternative, less-accessible devices.

The principle of the HemoSep® system is described in WO-A-2009/141589, and the processing bag used in the HemoSep® system is described in WO-A-2011/061533. The disclosures of both of those documents are hereby incorporated by reference, in their entirety.

The HemoSep® device salvages blood lost during high blood loss surgery, by concentrating the residual blood and recycling it so that it can be transfused back to the patient. The device consists of a blood bag which employs a superabsorbent material to absorb blood plasma and a mechanical agitator to concentrate blood sucked from the surgical site or drained from the heart-lung machine after the surgery. The separated cells are then returned to the patient by intravenous transfusion.

HemoSep® was designed as a haemoconcentration technology that produces a blood concentrate of all cell species, preserving the platelets, white blood cells and clotting residuals.

The process does not require centrifugation and associated blood transfer steps, as well as removing the need for highly trained technical personnel. The concept was based upon the use of a membrane-controlled superabsorber-driven plasma removal process, which is carried out in one vessel and requires no additional blood transfer steps or flushing procedures. Residual blood is introduced to the device, concentrated using the membrane-controlled superabsorber process and transfused back to the patient using a transfer bag. Another benefit of HemoSep® technology is that it produces a gelatinous waste product—essentially dilute plasma in a gel matrix—which is safer and easier to dispose of than the large volumes of fluid associated with more common centrifugation processes. The technique for removing the plasma from the blood product, leading to concentration of the cellular components, is fairly simple but involves a number of critical steps and controls.

The HemoSep® system comprises a blood reservoir to which blood is transported under suction from the wound site, and from which the blood is pumped to the HemoSep® bag. The HemoSep® bag is responsible for the removal of plasma from the blood, thereby producing a blood cell concentrate. To achieve this the HemoSep® bag contains an inner bag formed from a porous membrane that contains a superabsorbent material. The membrane has a pore size which prevents the migration of cellular species from the blood into the superabsorber section of the device. Free passage of the critically dilute plasma, however, is not restricted and as this fluid passes from the blood through the membrane into the superabsorber. This results in a concentration of the cellular components of the blood held within the bag. Once the appropriate level of haemoconcentration is achieved, the blood held in the bag is transferred into a transfer bag for subsequent transfusion to the patient.

In order to reduce the time required for absorption of plasma, and also to prevent the pores in the membrane becoming blocked by blood cells, the HemoSep® system further comprises a powered orbital shaker.

HemoSep® offers a wide range of benefits. It provides fast, simple cell concentration with improved patient outcomes:
Patient's own blood is transfused, reducing the risk of contamination and reaction
Decreased need for donor blood and associated transfusion products, leading to a reduction in donor dependency
Decreased blood loss
Assists in the reduction of post-operative bleeding, resulting in improved patient recovery
Maintenance of platelet population and associated preservation of normal clotting function
Removal of pro-inflammatory molecules, resulting in a reduction in post-operative complications
Reduction in donor demand
Improved patient recovery
Preservation of more normal clotting function
Reduction in post-operative complications

There are also profound advantages of using HemoSep® over current auto-transfusion cell-salvage systems, which are significantly more expensive in terms of initial equipment costs and cost of disposables, require specialist technicians and also remove viable platelets, clotting factors and plasma proteins essential to whole blood. In addition, the HemoSep® technology has a significantly smaller footprint and weight when compared to alternatives.

The HemoSep® system is simple, portable and easy to use. These characteristics are necessary for use in battlefield situations. However, the HemoSep® system also requires the use of a powered shaker to reduce processing time and to prevent clogging of the membrane pores, and a supply of suitable electrical power may not be available in a battlefield situation.

### BRIEF SUMMARY OF THE INVENTION

It is an object of the present invention to provide a field-deployable technology that allows recovery of cells that can be efficiently and quickly recovered to the patient. It is a further object of the invention to develop a convenient and elegant technology for military deployment where access to power and specialist personnel is often lacking, for routine application by non-specialists in an operational setting, where cells harvested by this device are directly transfused back into the casualty (autotransfusion), to offer critical primary intervention on the battlefield for haemorrhaging soldiers. It is a further object of the invention a device that requires no electrical power for the process for cell concentration or blood harvesting, and that can be operated by one member of the recovery team.

According to a first aspect of the invention, there is provided apparatus for blood processing, said apparatus comprising
a blood collection reservoir;
a blood collection conduit adapted and arranged to convey, in use, blood from a patient's wound site to the blood collection reservoir;
a blood transfer conduit adapted to convey, in use, blood from the blood collection reservoir to the patient or an intermediate blood processing unit; and
a manually operable pump unit comprising first and second peristaltic pumps, the first peristaltic pump being mounted about and acting upon the blood collection conduit, and the second peristaltic pump being mounted about and acting upon the blood transfer conduit,
wherein the pump unit is provided with an actuator adapted to switch the second peristaltic pump between an operative condition and an inoperative condition, in the operative condition the second peristaltic pump being engaged with the blood transfer conduit to convey blood from the blood collection reservoir while the first peristaltic pump is engaged with the blood collection conduit to convey blood from a wound site to the blood collection reservoir, and in the inoperative condition the second peristaltic pump being disengaged from the blood transfer conduit.

The first and second peristaltic pumps are preferably housed within a single common housing. The housing is preferably provided with a grip or handle by which it can be grasped by an operator in use.

Manual operation of the pump unit is preferably carried out by rotation of a handle that is connected to a suitable drive mechanism within the pump unit. The pump unit preferably includes gearing to increase the rate of rotation of the peristaltic pumps in proportion to the rate of rotation of the handle. Typically, the gearing ratio (ie the rate of rotation of the pumps relative to the rate of rotation of the handle) is from 1.5:1 to 10:1, more typically from 2:1 to 4:1.

Preferably, the flow rate of the first peristaltic pump is greater than 1 L/min, more preferably greater than 2 L/min or greater than 5 L/min or greater than 10 L/min. The flow rate of the second peristaltic pump, on the other hand, will generally be lower than that of the first peristaltic pump and is preferably less than 2 L/min, or less than 1 L/min or less than 0.5 L/min. The flow rates of the first and second peristaltic pumps may be in a ratio of between 2:1 and 10:1, more typically between 4:1 and 8:1. The flow rates of the pumps may be adjusted by appropriate selection of dimensions for the pumps and the respective conduits.

The first and second peristaltic pumps are preferably of generally conventional configuration, comprising sets of rollers (typically three in number) that orbit around the main axis of the pump and are periodically brought into engagement with, and at least partially occlude, the conduit with which the pump is associated.

The blood collection conduit and the blood transfer conduit are typically flexible tubes, most commonly of plastics material. The internal diameter of the blood collection conduit may be between about 5 mm and about 20 mm, more typically between about 10 mm and 15 mm. The internal diameter of the blood transfer conduit may be between about 2 mm and about 10 mm, more typically between about 3 mm and about 8 mm.

The blood collection conduit preferably terminates in a suction wand or the like of generally conventional form, the tip of the wand being inserted, in use, into blood at the wound site. Preferably, a supply of anti-coagulant is provided, the anti-coagulant being entrained in the collected blood in a manner that is known per se.

The actuator may take any suitable form effective to engage or disengage the second peristaltic pump.

In a first group of embodiments, in the inoperative condition the rollers of the second peristaltic pump are disengaged from the drive mechanism by which they are rotated in the operative condition. In such embodiments, the actuator has the nature of a clutch mechanism.

In another group of embodiments, the rollers of the second peristaltic pump are caused to rotate even in the inoperative condition, ie they remain coupled to the drive mechanism in that condition, but in the inoperative condition the rollers are displaced from the blood transfer conduit. In such embodiments, the actuator may be a push button or the like, depression of which brings the rollers into engagement with the conduit, and release of which causes the rollers to be released from engagement with the conduit.

Whilst the pump unit of the system of the invention is manually operable, such that it can be operated in the absence of electrical power, the pump unit is preferably configured such that where electrical power is available, it can be engaged with a supply of such power. To that end, the manually operable handle of the pump unit may be detachable to allow the pump unit to be mounted on a docking station or the like with an electrically driven spindle that engages the drive mechanism of the peristaltic pumps. The source of electrical power is most preferably a source of DC power.

Blood harvested from a wound site and conveyed to the blood collection reservoir by the first peristaltic pump will generally be in the form of a blood/air mixture that is very turbulent and prone to foaming. The flow needs to be made more laminar, without causing haemolysis. To achieve this, the invention further provides a defoaming device of novel construction. Such a defoaming device comprises an upstream, generally hemispheroidal funnel having an opening at the base thereof, and a downstream cup of complementary form to the funnel, juxtaposed surfaces of the funnel and the cup (ie the downstream or underside surface of the funnel and the upstream or upper surface of the cup) being spaced apart to form a gap, such that blood entering the funnel and passing through the opening in the funnel into the cup flows in random directions over the surface of the cup and flows out of the defoaming device through the gap.

The blood collection reservoir preferably further comprises a filter, downstream of the defoaming device, that is effective for removal of particulate matter, eg particles having a size greater than about 50 μm. Such a filter may take the form of a fabric tube depending from the defoaming device and that is closed at its distal end. Such a tube may contain a defoaming sponge to further reduce the occurrence of foaming.

An intermediate blood processing unit is preferably adapted to produce a red blood cell concentrate by removal of plasma from the blood conveyed to it. The blood processing unit may therefore be generally of the form of the known HemoSep® bag described above. In such a bag, blood plasma passes across a porous membrane and is absorbed by a superabsorbent material. Blood cells, however, are unable to pass through the membrane.

As also described above, however, the HemoSep® system utilises an electrically powered shaker to reduce the time required for haemoconcentration of the blood. Such a system is incompatible with the situations in which the present invention is envisaged to be of greatest utility, ie situations in which electrical power is unavailable. Surprisingly, however, it has been found that clinically satisfactory levels of haemoconcentration (ie sufficiently high levels of packed cell volume) may be achieved in acceptably short periods of time, without shaking, if the number and density of the pores in the membrane is increased substantially, such that the effective open area of the membrane is considerably increased. It has been found that despite vast increases in the number of pores, the membrane retains sufficient integrity to function as an effective barrier to blood cells.

Thus, according to a further aspect of the invention, there is provided a blood processing unit for the haemoconcentration of blood, which unit comprises a porous filter membrane having pores with an average size of less than 5 μm and wherein the membrane has an effective open area of at least 25%.

Preferably, the effective open area of the membrane is at least 30% or at least 40%.

By “effective open area” is meant the aggregate area of the pores present in the membrane relative to the overall area of the membrane.

Preferably, the pores in the membrane have an average size of less than 2 μm.

The system of the invention may further comprise a blood transfusion reservoir, typically a transfusion bag of conventional form, to which processed blood may be transferred from the blood processing unit.

### BRIEF DESCRIPTION OF THE DRAWINGS

The invention will now be described in greater detail, by way of illustration only, with reference to the accompany drawings, in which

FIG. 1 is a schematic view of a blood salvage and autotransfusion system in accordance with the invention;

FIG. 2 is a schematic view, partially cut away, of a manually operated pump forming part of the system of FIG. 1;

FIG. 3 shows (a) a side view, (b) a view from above, and (c) an exploded view, of a working embodiment of a pump of the form depicted schematically in FIG. 2;

FIG. 4 is a diagrammatic view of an actuation mechanism forming part of the pump of FIG. 3;

FIG. 5 shows (a) a perspective view of a docking station for the electrical operation of the pump of FIG. 3, and (b) another view of the docking station together with the pump, prior to engagement of the pump with the docking station;

FIG. 6 shows (a) a blood collection bag forming part of the system of FIG. 1 and—separately—a defoaming device and a polyester sock that are fitted to the blood collection bag, (b) a side view of the blood collection bag with the defoaming device and sock fitted, and (c) a plan view of the blood collection bag with the defoaming device and sock fitted;

FIG. 7 shows views of the defoaming device of FIG. 6, in particular (a) a perspective view from below of an outer component of the defoaming device, (b) a further perspective view of the outer component, from a different angle, (c) a perspective view from above of an inner component of the defoaming device, and (d) a perspective view of the defoaming device with the inner and outer components engaged;

FIG. 8 is a schematic side view of the defoaming device of FIG. 7, generally along the arrow X in FIG. 7(d), together with a disc by which the defoaming device is attached to a polyester sock and a tube connector disc;

FIG. 9 is a plan view of a blood processing bag forming part of the system of FIG. 1;

FIG. 10 is a cross-sectional view of the blood processing bag of FIG. 9, on the line A-A in FIG. 9;

FIG. 11 shows scanning electron micrograph (SEM) images of a membrane used in the construction of the blood processing bag of FIGS. 9 and 10, at (a) 1000× and (b) 3000× magnification, showing the porosity of the membrane; and

FIG. 12 is a plot comparing the performance of a blood processing bag constructed using the membrane of FIG. 11, in comparison to a prior art blood processing bag.

### DETAILED DESCRIPTION OF THE INVENTION

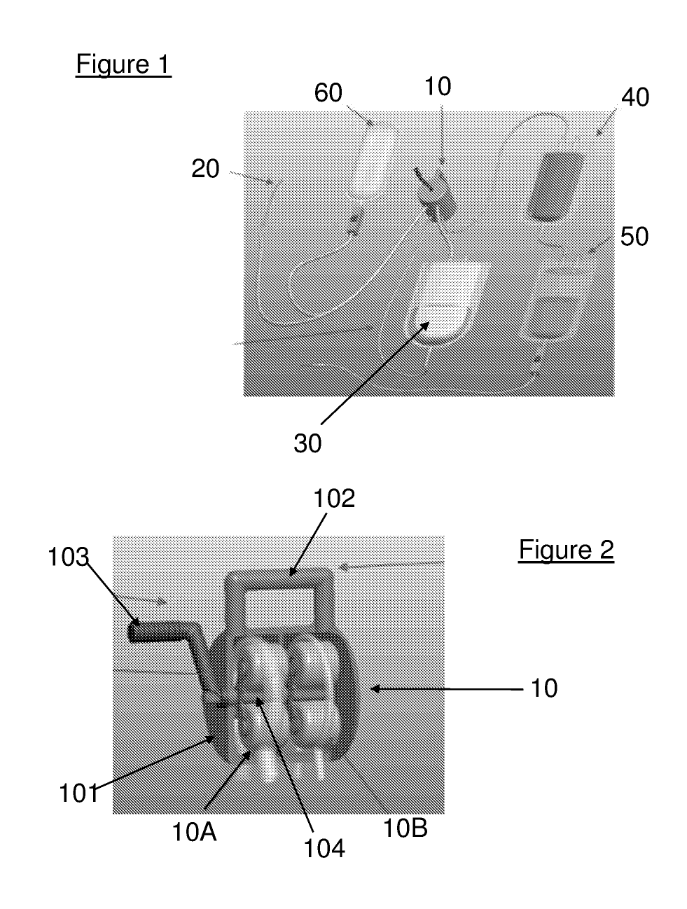

Referring first to FIG. 1, a blood salvage and autotransfusion system according to the invention comprises the following principal components:
manually operable peristaltic pump unit 10
suction wand 20
blood collection bag 30
blood processing bag 40
blood transfusion bag 50
anti-coagulant reservoir 60

The above components are connected by suitable conduits that are of generally conventional form and construction, typically being flexible tubes of plastics material fitted with suitable connectors for connection to the respective components.

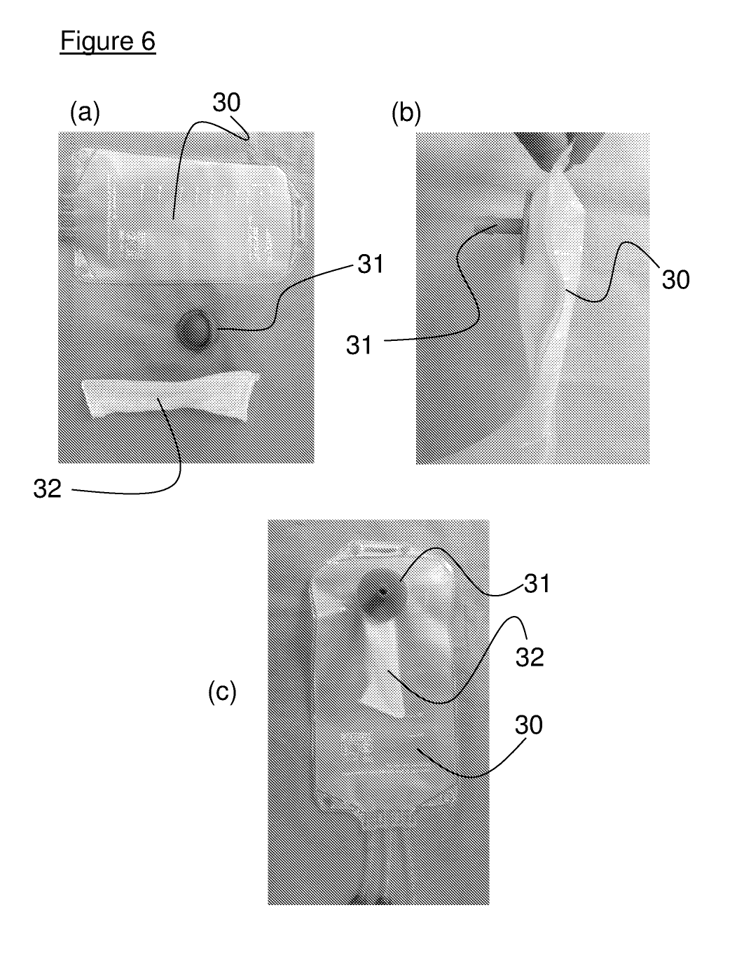

In use, the tip of suction wand 20 is inserted into blood at a wound site. As described in greater detail below, operation of the peristaltic pump unit 10 draws blood from the wound site to the blood collection bag 30. The fluid drawn from the wound site is a turbulent blood/air mixture. All air and major contaminants (eg clots, bone fragments etc) must be removed before the blood can be retransfused to the patient; blood collection bag 30 must perform that task efficiently without causing significant haemolysis. A defoaming device and other measures to achieve that objective are described below with reference to FIGS. 6 to 8.

Anti-coagulant is simultaneously drawn from anti-coagulant reservoir 60 and entrained in the blood, in a conventional manner. It should be noted, however, that anti-coagulant will not be needed where the wound is a chest wound, as shed blood from the chest is missing key coagulation factors and will not clot. The primary function of the anti-coagulant is then to boost the oxygen-carrying capacity of the patient's blood. Treatment with anti-coagulant will generally be valuable for blood shed from other wound sites and where the processed blood is intended to be stored, rather than immediately retransfused back to the patient.

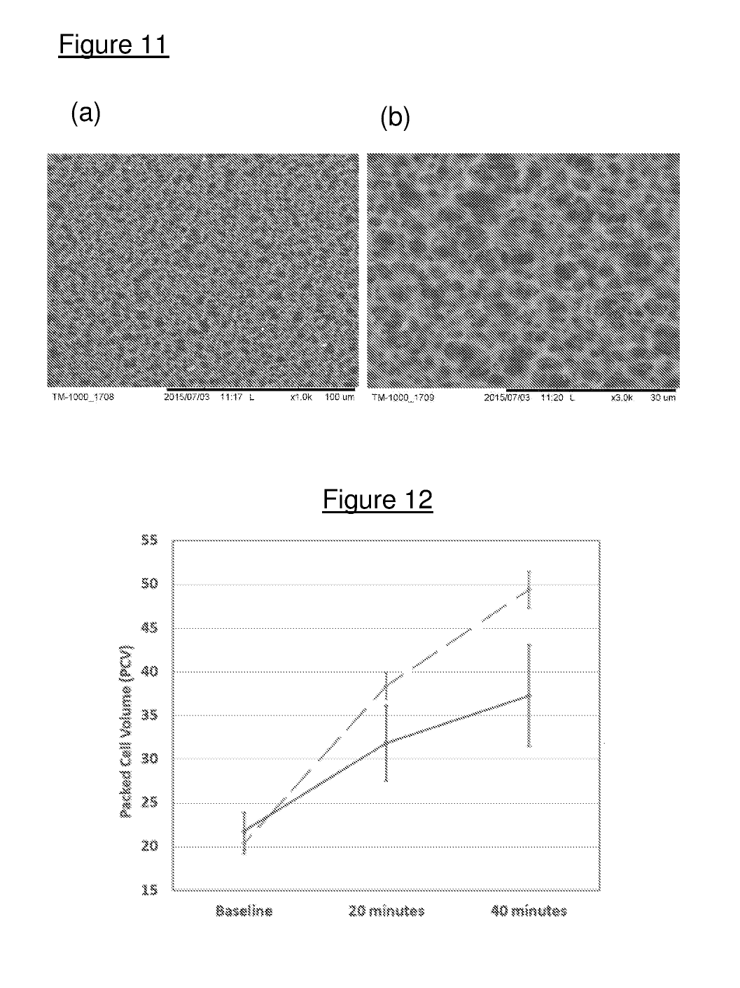

As also described in greater detail below, peristaltic pump unit 10 can be operated to draw blood from blood collection bag 30 and transfer it to blood processing bag 40. Blood processing bag 40 is, apart from certain inventive modifications that are described with reference to FIGS. 11 and 12, similar in form to the processing bag disclosed in WO-A-2011/061533, the teaching of which is hereby incorporated by reference in its entirety.

Blood processing bag 40 serves to remove plasma from the blood introduced into it, so producing a red blood cell concentrate suitable for transfusion back to the patient from whom the blood has been taken. Thus, when the desired degree of concentration has been achieved, the red blood cell concentrate is drained from blood processing bag 40 to transfusion bag 50, which is of generally conventional form and will not be described in any greater detail.

In some cases, where autotransfusion of blood back to the patient is urgent, blood processing bag 40 may be omitted from the system.

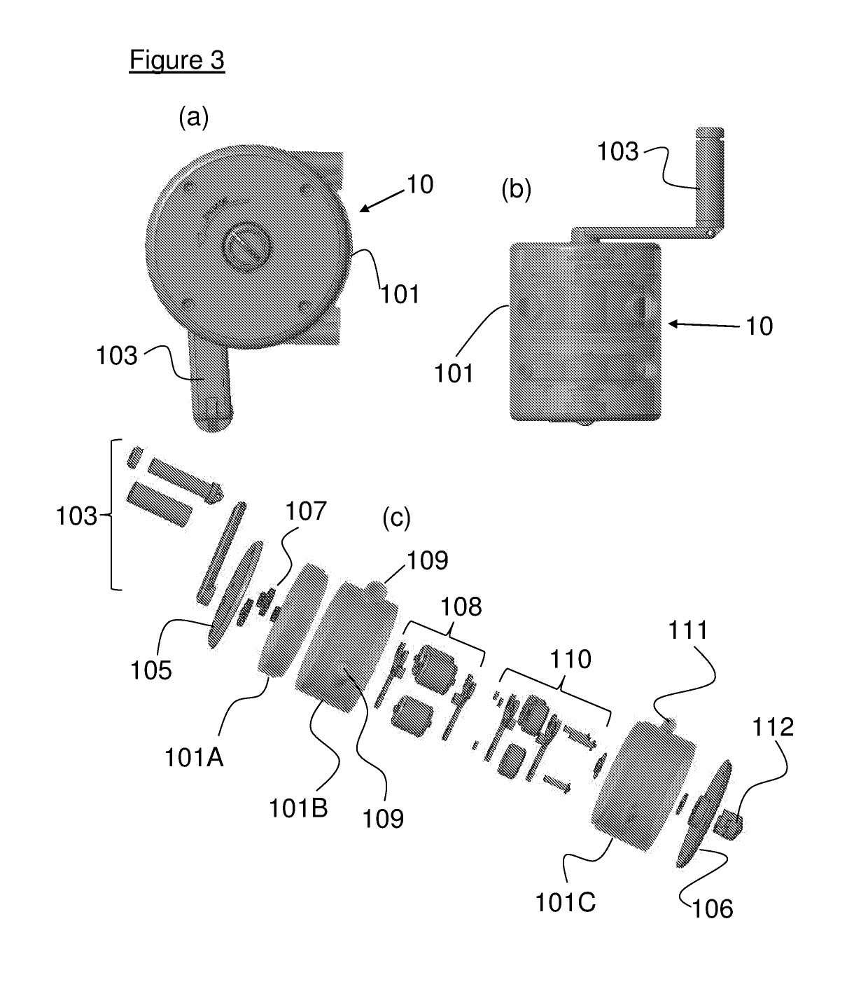

Turning now to FIGS. 2 and 3, peristaltic pump unit 10 is lightweight, compact and manually operable, such that it is suitable for deployment in battlefield situations, or other scenarios in which electrical power is not available.

As illustrated schematically in FIG. 2, peristaltic pump unit 10 comprises a generally cylindrical housing 101 having an integrally formed grip 102 by which pump unit 10 may be held be an operator during use. A cranked, detachable handle 103 is connected to a drive axle 104 mounted centrally within housing 101, and is used by the operator to manually drive the pumps contained within pump unit 10.

Housing 101 accommodates two separate peristaltic pumps 10A,10B, each of which is based upon a conventional peristaltic roller mechanism. Each such mechanism comprises a set of three rollers that are held in fixed relation to each other and which orbit about an axis (corresponding to the axis of drive axle 104). The rollers periodically bear against, and occlude, a flexible tube that extends through pump unit 10, between the rollers and the internal wall of housing 101.

Pump 10A is capable of relatively high flow (typically >10 L/min), which the other pump 10B has a relatively low flow configuration (typically <500 mL/min). The two pumps 10A,10B are connected to common drive axle 104, but operate independently using an actuation mechanism (not visible in FIG. 2) to activate the low flow pump 10B when required. The rollers of high flow pump 10A act upon a relatively large bore tube that extends from suction wand 20 to blood collection bag 30, while the rollers of low flow pump 10B act upon a relatively small bore tube that connects blood collection bag 30 with blood processing bag 40.

The high flow pump 10A can be operated constantly to evacuate blood from the patient to the blood collection bag 30, and operation of the low flow pump 10B does not interfere with that function. The drive axle 104 is manually rotated by means of cranked handle 103, with gearing (not visible in FIG. 2) enabling easy attainment of a suitable rotation speed. High flow pump 10A is capable of constantly extracting blood from the wound site while moving the blood into blood collection bag 30, but blood is drawn from blood collection bag 30 to blood processing bag 40 only when desired. That is achieved by means of the actuation mechanism, by means of which the rollers of low flow pump 10B are brought into engagement with the tube that connects blood collection bag 30 with blood processing bag 40, so pumping blood along that tube. When it is desired to cease operation of low flow pump 10B, the actuation mechanism is released, causing the rollers of low flow pump 10B to be disengaged from the tube, so stopping the pumping action of low flow pump 10B.

An embodiment of the pump illustrated schematically in FIG. 2 is shown in greater detail in FIG. 3, in which components corresponding to those described in relation to FIG. 2 are given the same reference numerals.

As described above, pump unit 10 comprises a generally cylindrical housing 101. A manually operable, cranked handle 103 is mounted on a central axis of housing 101.

As illustrated in FIG. 3(c), handle 103 has a multi-part construction, as is conventional for such a rotatable handle. As can also be seen in FIG. 3(c), housing 101 comprises three housing elements 101A,101B,101C, together with a pair of end plates 105,106. End plate 105 and housing element 101A are located adjacent to handle 103. Housing element 101A accommodates gearing 107 that controls the rate of rotation of the pump in relation to the the rate of rotation of handle 103, typically in a ratio of 2:1.

Housing element 101B, together with a first roller assembly 108 accommodated within it, constitutes high flow pump 10A. Housing element 101B is formed with a pair of tubular protrusions 109 through which passes the relatively large bore tube on which high flow pump 10A acts, such that when first roller assembly 108 is rotated within housing element 101B, the rollers of that assembly periodically bear against and occlude that tube, thereby generating a pumping action.

Similarly, housing element 101C and a second roller assembly 110 accommodated within it constitute the low flow pump 10B. Housing element 101C is formed with a pair of tubular protrusions 111 through which passes the relatively small bore tube on which low flow pump 10B acts. In the case of the low flow pump 10B, however, the rollers of the second roller assembly 110 bear against that tube—and so exert a pumping action—only when the low flow pump 10B is brought into operation by an actuator button 112.

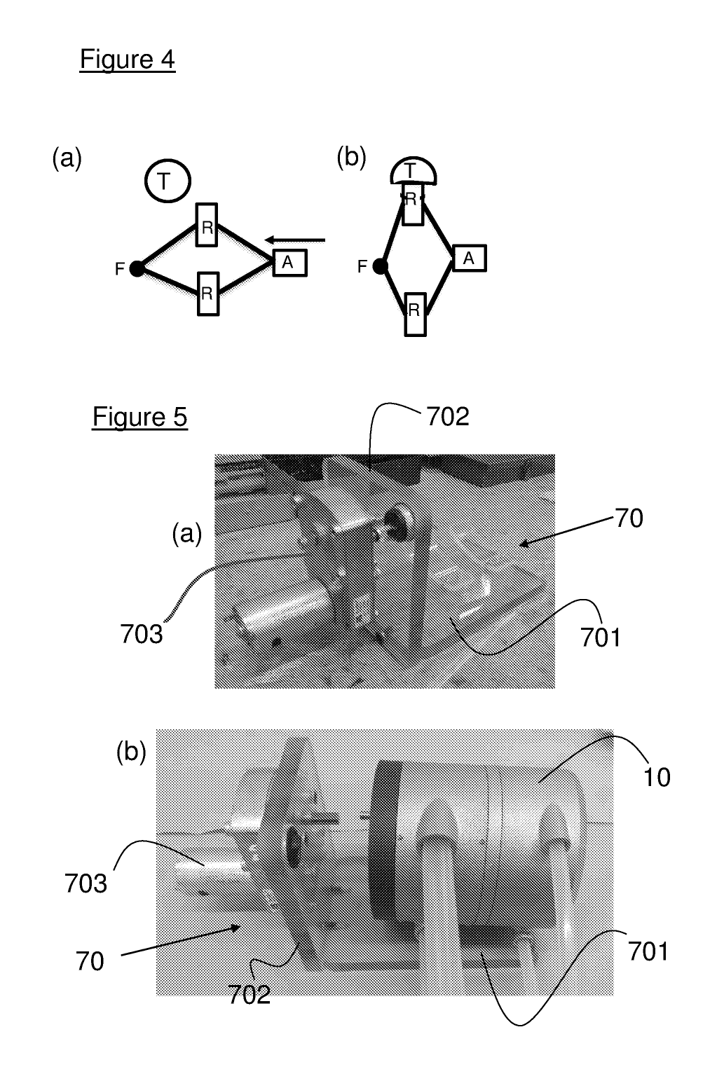

The manner in which the actuator button 112 operates is illustrated schematically in FIG. 4.

FIG. 4(a) shows diagrammatically the low flow pump 10B in an inoperative condition. The second roller assembly 110 is represented as a pair of rollers R mounted on spars having a parallelogram arrangement between a fixed point F and the actuator button A. The tube that passes through the low flow pump 10B is denoted T.

In the FIG. 4(a) arrangement, the rollers R are offset from tube T. Although the roller assembly rotates (about the axis extending between F and A), the rollers R exert no action upon tube T.

When it is desired to bring low flow pump 10B into operation, actuator A is depressed along the direction of the arrow in FIG. 4(a). Since the point F is fixed, the rollers R are displaced to the left (as viewed in FIG. 4(a)) and are also moved apart into the configuration shown in FIG. 4(b). In that operative configuration, the rollers R bear against—and, as they rotate, periodically occlude—tube T. Hence, in this configuration, the low flow pump 10B is actuated. When it is desired to cease operation of low flow pump 10B, actuator A is returned to its FIG. 4(a) position, thereby disengaging rollers R from tube T.

As described above, pump unit 10 is manually operable, and so is suitable for use in situations in which electrical power is not available. Where such power is available, however, it is clearly desirable for it to be used to operate pump unit 10. To achieve that objective, manually operable, cranked handle 103 of pump unit 10 is detached and instead pump unit 10 is engaged with docking station 70 of FIG. 4. Docking station 70 comprises a base 701 having suitable supports for housing 101 of pump unit 10 and an upstand 702 upon which is mounted a DC Geared Motor 703 (24 VDC 24 Nm 125 rpm, Mellor Electric, RS Components, UK). Motor 703 is coupled (Ruland Aluminium Flexible Beam Coupling, RS Components, UK) with the pump and powered with a DC power supply (Digimess Concept Series, Digimess Instruments Ltd, Derby, UK).

The pumping characteristics of pump unit 10 when engaged with docking station 70 were then tested using fresh bovine blood collected from a local abattoir on the morning of assessment. Flow rate was controlled by altering the voltage output of the power supply. Testing was performed with the motor running at a maximum rate of 84 rpm at 24V DC.

The following flow rates were achieved:

High flow pump flow rate: 3 L/min

Low flow pump flow rate: 600 mL/min

To assess whether the flow rates and turbulence would cause blood haemolysis, blood samples were taken before and after each flow test. High flow pump tests were performed using blood and a blood/air mix (to mimic blood evacuated from a wound site), and the low flow pump was tested using blood. Blood samples were placed into microhaematocrit capillary tubes (Brand GmbH+Co KG, Wertheim, Germany), and spun in a centrifuge at 2000 revolutions per minute for three minutes. Visual inspection showed that there was no evident haemolysis—red discolouration of plasma confirms presence of haemolysis—and this was corroborated with spectrophotometric analysis that showed only trace levels of haemolysis before and after pumping.

As noted above, the fluid drawn from the wound site is a turbulent blood/air mixture. All air and major contaminants (eg clots, bone fragments etc) must be removed before the blood can be retransfused to the patient; blood collection bag 30 must perform that task efficiently without causing significant haemolysis. To address that objective, blood collection bag 30 is fitted with a defoaming device and a polyester sock that will now be described with reference to FIGS. 6 to 8.

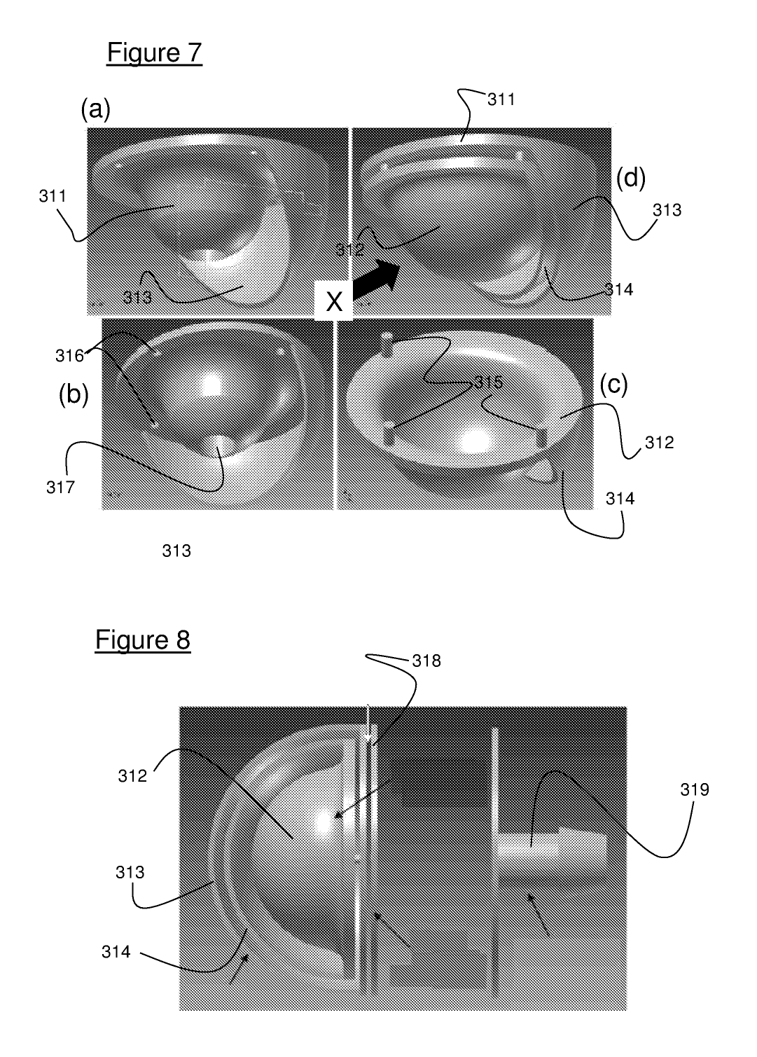

Turning first to FIG. 6, blood collection bag 30 is of broadly conventional form, but is fitted with defoaming device 31 that constitutes the inlet through which blood drawn from the wound site enters blood collection bag 30. The construction of defoaming device 31 and the manner in which it is fitted to blood collection bag 30 is described in greater detail below, with reference to FIGS. 7 and 8. A polyester sock 32 is fitted to defoaming device 31, within blood collection bag 30. Sock 32 comprises a tube of thinly woven polyester fibres that is closed at one end. The open end of the tube is fitted to the defoaming device 31, as described below, such that blood flowing into blood collection bag 30 through defoaming device 31 passes into the interior of sock 32 and then through the walls of sock 32 into blood collection bag 30. The spacing of the fibres from which sock 32 is woven means that sock 32 filters out of the blood particulates having a size greater than approximately 50-70 μm.

The blood/air mixture entering blood collection bag 30 is very turbulent and prone to foaming. The flow needs to be made more laminar, without causing haemolysis. This is achieved primarily by means of defoaming device 31, but to further reduce foaming sock 32 contains a polyester defoaming sponge that is coated with an anti-foaming agent, such as a hydrophobic silica dispersed in silicone oil.

Defoaming device 31 is shown in detail in FIGS. 7 and 8, and comprises two principal components: a funnel element 311 and a cup 312. Funnel 311 and cup 312 are both generally hemispherical in form with planar peripheral flanges and part-spherical hoods (313,314 respectively) extending from parts of those flanges. The flange of cup 312 is provided with three upstanding pins 315 that engage in corresponding openings 316 in the flange of funnel 311. Funnel 311 has a central opening 317.

In the assembled condition shown in FIG. 7(d), the cup 312 is fixed to the funnel 311, with the major surfaces of the two components being slightly spaced apart. Thus, a gap exists between the upper surfaces of cup 312 and the lower surfaces of funnel 311 and a similar gap exists between the juxtaposed surfaces of the respective hoods 313,314.

Defoaming device 31 is inserted into the open end of sock 32 and the edges of the mouth of sock 32 are folded over the flange of funnel 311 and held in place by a disc 318—see FIG. 8—that has a central opening. Disc 318 is fixed to the flange of funnel 311 by means of adhesive, thereby captivating the edges of sock 32 between disc 318 and defoaming device 31. The assembly of defoaming device 31 and sock 32 is inserted into blood collection bag 30 through a suitably shaped and dimensioned opening in the wall of blood collection bag 30 and fixed in place by attachment (by adhesive or ultrasonic welding or other suitable technique) of a tube connector 319 having a disc-shaped base. Tube connector 319 adheres to the flange of funnel 311 and captivates the edge of the opening in the wall of blood collection bag 30, thereby completing the assembly. Defoaming device 31 is oriented such that hoods 313,314 are positioned uppermost within blood collection bag 30. This assists with ensuring that sock 32 hangs vertically within blood collection bag 30 during use, and also helps to maintain an open configuration of sock 32.

The end of the conduit leading from pump unit 10 to blood collection bag 30 is fitted onto tube connector 319. In use, the turbulent blood/air mixture transported from the wound site to blood collection bag 30 by pump unit 10 passes through tube connector 391, through the central opening of disc 318 and into funnel 311. The mixture is channelled through the central opening 317 of funnel 311 into cup 312. The mixture then flows in random directions along the surface of cup 312, exiting defoaming device 31 and entering sock 32 through the gap between cup 312 and funnel 311.

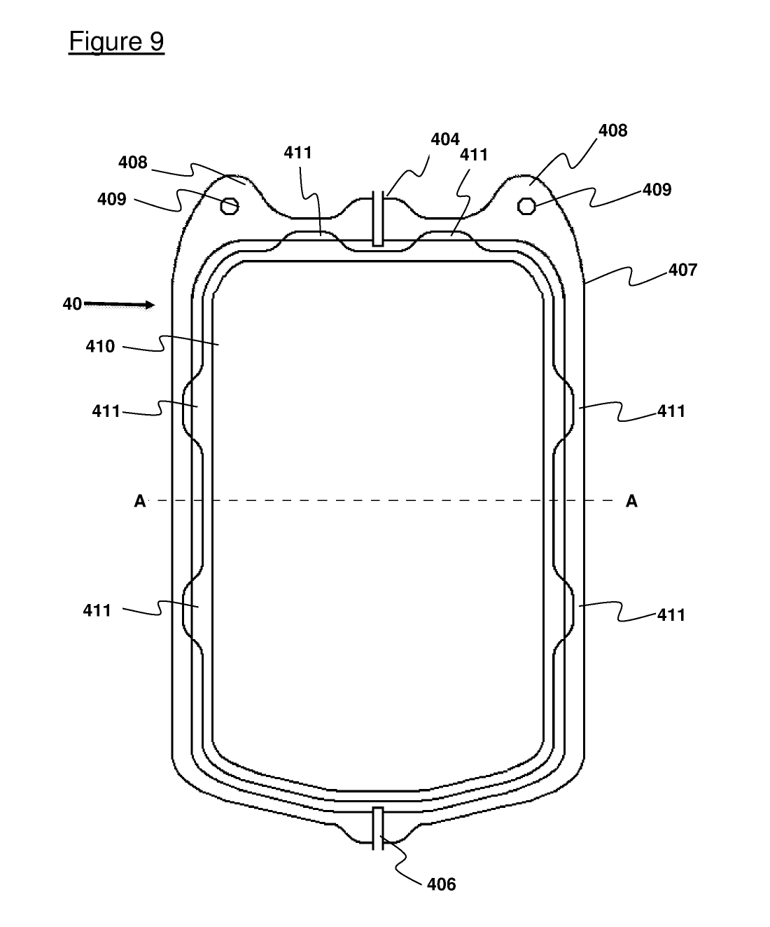

Finally, referring to FIGS. 9 to 12, there is shown the blood processing bag 40 that forms part of the system of FIG. 1.

Blood processing bag 40 comprises an outer bag 401 formed of a tough impermeable material, an inner bag 402 formed of a porous membrane material and contained within outer bag 401, and a superabsorbent material 403 encapsulated within inner bag 402. The outer bag 401 has an inlet port 404, through which blood may—by operation of the low flow peristaltic pump 10B of pump unit 10 causing it to be drawn from blood collection bag 30—pass into blood processing bag 40 and enter the cavity 405 formed between outer bag 401 and inner bag 402, and an outlet port 406, though which processed blood may exit blood processing bag 40 and be drained into transfusion bag 50.

Outer bag 401 is formed of polyvinylchloride (PVC) sheets and inner bag 402 is formed of porous polycarbonate membrane. Both outer bag 401 and inner bag 402 are formed by fastening two sheets of material together around their edges by heat welding. The material of outer bag 401 is impermeable to blood plasma, whereas the material of inner bag 402 permits blood plasma, but not blood cells suspended in the plasma, to pass through it and into the interior of inner bag 402. For instance, inner bag 402 is formed of material having pores with a maximum size of no greater than 5 μm, and more commonly 1-2 μm, to permit blood plasma, but not red blood cells, to pass through.

The area around the edge of outer bag 401 where the two polyvinylchloride (PVC) sheets are welded together defines a welded portion 407. This welded portion 407 projects from each upper corner of outer bag 401 to form extensions 408. Each extension 408 has an aperture 409 to allow blood processing bag 40 to be hung from a suitable support.

The area around the edge of inner bag 402 where the two porous polycarbonate membranes are welded together also defines a welded portion 410. The superabsorbent material 403 is entirely encapsulated by inner bag 402. The welded portion 410 around the edge of the inner bag 402 extends outwardly to form a number of tabs 411, which are trapped within the welded region 407 at the periphery of the outer bag 401 at a number of points to suspend the inner bag 402 within the outer bag 401.

To the extent described above, blood processing bag 40 is as described in WO-A-2011/061533. The manner in which blood processing bag 40 differs from that earlier disclosure, however, is illustrated in FIGS. 11 and 12.

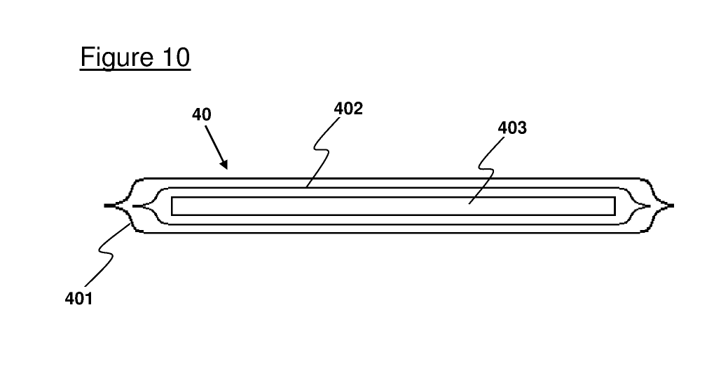

FIG. 10 shows SEM images of the porous membrane material used to form inner bag 402. The number and density of pores present in the membrane material is considerably increased, in comparison to that previously used, such that the effective open area (EOA) of the membrane is around 45%. For prior art membranes used for the same application, the EOA was only about 7%. Surprisingly, it has been found that membranes with such a high EOA retain sufficient structural integrity, whilst increasing the rate of passage of blood plasma across the membrane material and so reducing the time required for a given degree of blood concentration.

To assess the performance of inner bags 402 constructed using the high EOA membrane material of the invention, haemoconcentration efficacy was assessed using fresh bovine blood (n=10). To reduce the initial packed cell volume (PCV) before haemoconcentration assessment, the blood was diluted with saline (0.9 g sodium chloride per 100 ml). Baseline measures was recorded and the bags were primed with 100 ml of saline immediately before blood was introduced. Bags were filled with 350 ml of the blood-saline mix and placed on an orbital shaker. Blood samples were taken after 20 minutes and after 40 minutes. Blood samples were collected with microcapillary tubes and spun in a microhaematocrit centrifuge (Hawksley, Sussex, UK) for 3 minutes at 2000 rpm. Manual estimation of the haematocrit was performed using a Hawksley tube reader (Hawksley, England, UK). A comparison was made with historical files (n=19) relating to the bags made with conventional membrane material having an EOA of about 7%.

The results are shown in FIG. 11, where data are presented as the mean±SD. Results for high EOA bags are shown by the broken line; comparative historical data for low EOA bags is shown by the solid line.

Baseline PCV for the high EOA bags was 20.4±1.17%, which increased to 38.4±1.5% at 20 minutes processing and 49.4±2.1% at 40 minutes. Baseline PCV historical data (low EOA bags) was 21.8±2.15%, which increased to 31.9±4.3% at 20 minutes and 37.3±5.8% at 40 minutes. Thus, with the high EOA bags, PCV reached clinically acceptable levels in less than 20 minutes.

1. Apparatus for blood processing, said apparatus comprising:
a blood collection reservoir;
a blood collection conduit adapted and arranged to convey, in use, blood from a patient's wound site to the blood collection reservoir;
a blood transfer conduit adapted to convey, in use, blood from the blood collection reservoir to the patient or to an intermediate blood processing unit; and
a manually operable pump unit comprising first and second peristaltic pumps, the first peristaltic pump being mounted about and acting upon the blood collection conduit, and the second peristaltic pump being mounted about and acting upon the blood transfer conduit,
wherein the pump unit is provided with an actuator adapted to switch the second peristaltic pump between an operative condition and an inoperative condition, in the operative condition the second peristaltic pump being engaged with the blood transfer conduit to convey blood from the blood collection reservoir while the first peristaltic pump is engaged with the blood collection conduit to convey blood from a wound site to the blood collection reservoir, and in the inoperative condition the second peristaltic pump being disengaged from the blood transfer conduit.

2. Apparatus according to claim 1, wherein the first and second peristaltic pumps are housed within a single common housing.

3. Apparatus according to claim 2, wherein the housing is provided with a grip or handle by which it can be grasped by an operator in use.

4. Apparatus according to claim 1, wherein manual operation of the pump unit is carried out by rotation of a handle that is connected to a suitable drive mechanism within the pump unit.

5. Apparatus according to claim 4, wherein the pump unit includes gearing to increase the rate of rotation of the peristaltic pumps in proportion to the rate of rotation of the handle, for instance with a gearing ratio of from 1.5:1 to 10:1, more typically from 2:1 to 4:1.

6. Apparatus according to claim 1, wherein the flow rate of the first peristaltic pump is greater than 1 L/min, more preferably greater than 2 L/min or greater than 5 L/min or greater than 10 L/min.

7. Apparatus according to claim 1, wherein the flow rate of the second peristaltic pump is lower than that of the first peristaltic pump and is less than 2 L/min, or less than 1 L/min or less than 0.5 L/min.

8. Apparatus according to claim 7, wherein the flow rates of the first and second peristaltic pumps are in a ratio of between 2:1 and 10:1, more typically between 4:1 and 8:1.

9. Apparatus according to claim 1, wherein the first and second peristaltic pumps comprise sets of rollers that orbit around the main axis of the pump and are periodically brought into engagement with, and at least partially occlude, the conduit with which the pump is associated.

10. Apparatus according to claim 1, wherein the blood collection conduit and the blood transfer conduit are flexible tubes, most commonly of plastics material.

11. Apparatus according to claim 1, wherein the internal diameter of the blood collection conduit is between about 5 mm and about 20 mm, more typically between about 10 mm and 15 mm.

12. Apparatus according to claim 1, wherein the internal diameter of the blood transfer conduit is between about 2 mm and about 10 mm, more typically between about 3 mm and about 8 mm.

13. Apparatus according to claim 1, wherein the blood collection conduit terminates in a suction wand or the like, the tip of the wand being inserted, in use, into blood at the wound site.

14. Apparatus according to claim 1, wherein a supply of anti-coagulant is provided.

15. Apparatus according to claim 1, wherein in the inoperative condition the rollers of the second peristaltic pump are disengaged from the drive mechanism by which they are rotated in the operative condition.

16. Apparatus according to claim 1, wherein the rollers of the second peristaltic pump are caused to rotate even in the inoperative condition, ie they remain coupled to the drive mechanism in that condition, but in the inoperative condition the rollers are displaced from the blood processing conduit.

17. Apparatus according to claim 1, wherein a manually operable handle of the pump unit is detachable to allow the pump unit to be mounted on a docking station or the like with an electrically driven spindle that engages the drive mechanism of the peristaltic pumps.

18. Apparatus according to claim 17, wherein the source of electrical power is a source of DC power.

19. Apparatus according to claim 1, wherein an intermediate blood processing unit is adapted to produce a red blood cell concentrate by removal of plasma from the blood conveyed to it.

20. A defoaming device comprising an upstream, generally hemispheroidal funnel having an opening at the base thereof, and a downstream cup of complementary form to the funnel, juxtaposed surfaces of the funnel and the cup (ie the downstream or underside surface of the funnel and the upstream or upper surface of the cup) being spaced apart to form a gap, such that blood entering the funnel and passing through the opening in the funnel into the cup flows in random directions over the surface of the cup and flows out of the defoaming device through the gap.

21. Apparatus according to claim 1, wherein the blood collection reservoir comprises a defoaming device.

22. Apparatus according to claim 21, wherein the blood collection reservoir further comprises a filter, downstream of the defoaming device, that is effective for removal of particulate matter, eg particles having a size greater than about 50 μm.

23. Apparatus according to claim 22, wherein the filter takes the form of a fabric tube depending from the defoaming device and that is closed at its distal end, and optionally containing a defoaming sponge to further reduce the occurrence of foaming.

24. A blood processing unit for the haemoconcentration of blood, which unit comprises a porous filter membrane having pores with an average size of less than 5 μm and wherein the membrane has an effective open area of at least 25%.

25. A blood processing unit according to claim 24, wherein the effective open area of the membrane is at least 30% or at least 40%.

26. A blood processing unit according to claim 24, wherein the pores in the membrane have an average size of less than 2 μm.

27. Apparatus according to claim 1, wherein the blood processing unit is a blood processing unit.
